(19) United States
(12) Patent Application Publication
(10) Pub. No.: US 2025/0281558 A1
(43) Pub. Date: Sep. 11, 2025

(54) Composition and Method for Treating Colic in Infants

(71) Applicant: Ddrops Company, Woodbridge (CA)

(72) Inventor: Simon Vieth, Toronto (CA)

(73) Assignee: Ddrops Company, Woodbridge, ON (CA)

(21) Appl. No.: 19/071,113

(22) Filed: Mar. 5, 2025

Related U.S. Application Data
(60) Provisional application No. 63/561,858, filed on Mar. 6, 2024.

(51) Int. Cl. A61K 36/23; A61K 9/00; A61K 9/08; A61K 36/235; A61K 47/14

(52) U.S. Cl. CPC A61K 36/23; A61K 9/0056; A61K 9/08; A61K 36/235; A61K 47/14

(57) ABSTRACT

The present invention is both a composition and a method for treating colic in infants. The present invention provides a composition and method that addresses colic symptoms in a multi-faceted way, specifically by administering a physiologically-effective dose of anethole, a smooth muscle antispasmodic, in a form that additionally provides pain relief via sweet analgesia and non-nutritive sucking.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD OF THE INVENTION

The invention is both a composition and method for treating colic in infants.

### BACKGROUND

Treatments of colic are known in the art. They all have significant drawbacks.

Colic is poorly characterized and many theories exist regarding its cause, and therefore complicate developing effective treatments.

Some theorize it may be attributed to an infant's inability to digest milk proteins, mom's ingestion of allergens, or an underdeveloped gut.

Colic is generally diagnosed if the Wessel criteria are met, namely the ‘rule of threes’ in healthy, well-fed infants: three hrs crying, three days per week, over three weeks.

Others theorize there may be a number of ‘colic syndromes’ at play, each with their own distinct mode of action. This suggests that colic is a pattern of behavior attributed to a complex interplay of gastrointestinal, developmental, and possibly psychological factors.

Barring convenient or plausible interventions for other factors, most colic interventions aim to generally address stomach discomfort.

### Gripe Water

Colic has been treated using ‘gripe water’ (“GW”) for over a hundred years.

GW is a folk remedy that does not have a universal formation and has had an evolving and inconsistent composition. GW acts more as a traditional concoction of ingredients, only some of which having a plausible mode of action, (e.g., herbal ingredients) while most main ingredients likely just placate and generally soothe the child (e.g., alcohol; sweet tasting ingredients like glycerine).

One GW is composed of water, glycerine, emulsifier (i.e, Polysorbate 80), herbal extracts, flavors, and preservatives.

It utilizes non-standardized amounts of raw and extracted herbal ingredients. The extracts typically are powder extracts that are primarily herbal constituents.

GW utilizes a large volume per dose (5 mL+) with an even larger volume dosed per day if given multiple doses as directed (30 mL+) relative to the size of the infant.

GW is usually a water based formula requiring antimicrobial preservative, emulsifying surfactant, and other excipients that do not contribute to treatment of symptoms.

GW has dubious clinical merit and might function entirely as placebo or as “sweet analgesia.”

Herbal extracts tend to be the most common medicinal ingredients in modern GW products, including extracts of dill, fennel, ginger, chamomile. There is little clinical evidence of efficacy of these raw herbs and their extracts in relieving stomach discomfort.

Many GWs contain non-standardized herbal ingredients or their extracts. Standardization aims to ensure a standard equivalent of the raw herb, not any of its constituents specifically. Herbal extracts in GW are typically macerated raw herb (issues: inconsistent composition, influenced by growing region, season), insoluble extracts (issues: mainly fiber components of the herb which have no clinical merit for colic relief), or water-soluble extracts (issue: including little or no anethole, the sole component of fennel or anise clinically associated with gastrointestinal discomfort relief).

Another common GW active ingredient is baking soda, which acts to reduce stomach acidity and gastric reflux. This is only effective if colic manifests because of excess stomach acids, which itself is an unsupported cause. Baking soda also serves to reduce acidity by increasing pH while also increasing stomach gaseousness. This might be a plausible cause of infant gastrointestinal discomfort, and a symptom pharmaceutical interventions aim to eliminate.

GW that include pharmaceutical treatments typically aim to either reduce pressure build-up in the stomach, or relax the gastrointestinal system. For example, simethicone, an anti-foaming agent, acts to reduce stomach gases. This is effective only if the colic cause is gastrointestinal gas build up and pressure.

A liquid preparation of simethicone with fennel oil and dill is a common colic treatment in India. Fennel oil included is in this large-volume emulsion (˜0.3 mL per dose), but only delivering 0.21 mg fennel oil per dose, meaning the treatment is principally sucrose solution with simethicone, and is not a fennel oil treatment.

Dicyclomine is another drug used in the past to treat colic in GW. It alleviates intestinal spasms and but has been known to cause severe side effects in infants. Thus, it is no longer supported for use in treatment of colic.

### Shushunov Fennel Oil Emulsion

The Shushonov fennel oil emulsion is described in U.S. Pat. No. 6,703,022 to Shushonov.

It employs a large volume per dose (5 mL+). It is limited to fennel seed only, and does utilize any other anethole-rich essential oils.

It requires an emulsifying agent at a high volume relative to small quantity of fennel seed.

The essential oil effectively eliminates the sweetness imparted by anethole, negating the ‘sweet analgesia’ effect that the herbal ingredient would otherwise induce.

The Shushunov patent referenced the Weizman et al. 1993 study of herbal teas (chamomile, verbena, licorice, fennel, balm-mint) but failed to fully address the large volume issue.

The Shushonov fennel oil emulsion fails to reduce dose volume fully by eliminating water and emulsifying agent and replacing them with a small volume ('drop') of nutritional liquid oil.

### Gripe Water “Gel”

The Grip Water Gel (“GW Gel”) utilizes a large 3.3g dose. It is dispensed inaccurately instructing to administer “a pea-sized amount.”

It is comprised of raw fennel seed, and not a concentrated and standardized extract. It utilizes a water-based, glycerine sweetened composition.

There is a need for an improved treatment of colic in infants which reduces the overall volume and dose. The present invention is not disclosed in the prior art.

### SUMMARY OF THE INVENTION

The invention is both a composition and a method for treating colic in infants.

The present invention provides a composition and method that addresses colic symptoms in a multi-faceted way, specifically by administering a physiologically-effective dose of anethole, a smooth muscle antispasmodic, in a form that additionally provides pain relief via sweet analgesia and non-nutritive sucking.

One embodiment of the present invention is a composition of concentrated oil-based solution of essential oil of fennel seed or anise seed in medium chain triglyceride (MCT) oil.

The composition and method together treat colic symptoms in a unique, three-fold approach.
1) The present invention utilizes anethole, which acts as a smooth-muscle antispasmodic to relieve gastrointestinal cramping and discomfort.
2) The present invention utilizes a concentrated slightly sweet anethole solution is administered directly to the infant providing a ‘sweet analgesia’ effect; and,
3.) The present invention uses a clinically-effective dose of anethole in a sufficiently small volume so as to accommodate administration of a single drop from a surface. This encourages non-nutritive sucking, a clinically-proven non-pharmacological method of pain-relief.

In a preferred embodiment, the anethole is administered with essential oils. Non-limiting examples of such include the essential oils selected which are most highly comprised of ‘anethole’ (e.g., fennel seed oil is 65-75% anethole; anise seed oil is 80-95% anethole).

Anethole (IUPAC 1-Methoxy-4-[(E)-prop-1-enyl]benzene; cis- and trans-isomers exist, trans-anethole) is preferred for use herein. This is an oil-soluble phenylpropanoid that has a sweetness approximately thirteen times greater than sucrose and is clinically demonstrated to be a smooth-muscle antispasmodic.

As used herein, a dose refers to one drop of oil solution weighing approximately 26 mg., which is clinically effective. An example of a dosing regimen would be 1-2 drops, administered up to 5 times per day.

The essential oils are standardized to 65-95% anethole content prior to blending. The blended oil solution is comprised of either fennel or anise seed essential oil at concentrations sufficiently high so as to deliver 1.5-3 mg to 1-5 mg anethole per dose.

The present invention also includes the method for administering the composition that provides a multi-faceted treatment of colic in infants. The composition is administered in its concentrated form directly into the infant's mouth, either drop wise or by first applying the drop to a clean surface such as a spoon, pacifier or a washed finger, and the infant is allowed to suck the composition from the surface.

In a preferred embodiment, the composition is dispensed in drops from a bottle fitted with an orifice reducing dispenser. However, a conventional bulb-style dropper may also be used. In a preferred embodiment, the bottle is an amber glass tincture bottle or euro dropper.

In a preferred embodiment, the composition includes fennel seed oil as the source of anethole. In a preferred embodiment includes an oil blend of ˜9.4% fennel seed oil, itself comprised 73% of anethole.

In another preferred embodiment, the composition includes anise seed oil as the source of anethole.

In a preferred embodiment, the present invention utilizes a medium chain triglyceride (“MCT”) oil to suspend the solution. Non-limiting examples of such MCT oils include vegetable oil, olive oil, or any other similarly-viscous, shelf-stable, minimally-flavored nutritional oil.

Examples of various compositions of the present invention are detailed below.

### EXAMPLE ONE

In one embodiment, fennel seed essential oil is administered to an infant for the treatment of colic symptoms. More specifically, it consists of 2.5 mg fennel seed essential oil suspended in 0.027 ml of an MCT oil. The solution contains 1.825 mg of anethole. The method of administration is application to the finger, and allowing the baby to suck. As it contains anethole, this solution enables a smooth muscle anti-spasmodic effect and sweet analgesia for the comfort of the infant.

### EXAMPLE TWO

In another embodiment, anise seed essential oil is administered to an infant for the treatment of colic symptoms. More it consists of 2.5 mg anethole seed essential oil suspended in 0.027 ml of an MCT oil. The solution contains 2.375 mg of anethole. The method of administration is application to the finger, and allowing the baby to suck. As it contains anethole, this solution enables a smooth muscle anti-spasmodic effect and sweet analgesia for the comfort of the infant.

One of skill in the art would appreciate that the present invention can be modified as follows, depending on the response from the infant.
(1) Using different concentrations of each oil in the oil blend.
(2) Adding other stomach discomfort-relieving oils into the blend (e.g., ginger oil, dill oil, and/or chamomile oil).

While several possible embodiments of the invention have been described above and illustrated in some cases, it should be interpreted and understood as to have been presented only by way of illustration and example, but not by limitation. Thus, the breadth and scope of a preferred embodiment should not be limited by any of the above-described exemplary embodiments.

It will be apparent to those skilled in the art that other embodiments of the invention will be apparent to those skilled in the art from consideration of the specification and practice of the invention. While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope of the invention. It is intended that the specification and examples be considered as exemplary, with the true scope of the invention being indicated by the claims.

We claim:

1. A composition comprising:
a solution of a medium chain triglyceride oil; and,
an anethole containing agent.

2. The composition of claim 1 wherein the anethole containing agent weighs between 2.0-3.0 mg per dose.

3. The composition of claim 2 wherein the anethole containing agent weighs 2.5 mg per dose.

4. The composition of claim 3 wherein the anethole containing agent is either anise seed essential oil or fennel seed essential oil.

5. The composition of claim 4 wherein the solution of a medium chain triglyceride oil has a volume between 0.025-0.75 ml per dose.

6. The composition of claim 5 wherein the medium chain triglyceride oil is vegetable oil.

7. A composition comprising:
a solution of a medium chain triglyceride oil;
an anethole containing agent;
an adjunct sweetener; and,
an adjunct essential oil selected from a group consisting of ginger, chamomile, and lemon balm.

8. The composition of claim 7 wherein the anethole containing agent weighs between 2.0-3.0 mg per dose.

9. The composition of claim 8 wherein the anethole containing agent weighs 2.5 mg per dose.

10. The composition of claim 9 wherein the anethole containing agent is either anise seed essential oil or fennel seed essential oil.

11. The composition of claim 11 wherein the solution of a medium chain triglyceride oil has a volume between 0.025-0.75 ml per dose.

12. The composition of claim 12 wherein the medium chain triglyceride oil is vegetable oil.

13. A method of treatment of the symptoms of colic in an infant that utilizes the composition of claim 1, consisting of the following steps:
In a first step, applying up to three drops of said composition to an object;
In a second step, inserting said object into a mouth of said infant and orally administering said composition to said object; and,
In a third step, having said infant suck on said object.

14. The method of claim 13 wherein the anethole containing agent weighs between 2.0-3.0 mg.

15. The method of claim 14 wherein the anethole containing agent is either anise seed essential oil or fennel seed essential oil.

16. The method of claim 15 wherein the solution of a medium chain triglyceride oil has a volume between 0.025-0.75 ml.

17. The method of claim 16 wherein the medium chain triglyceride oil is vegetable oil.
